(19) United States
(12) Patent Application Publication
(10) Pub. No.: US 2011/0082385 A1
(43) Pub. Date: Apr. 7, 2011

(54) METHOD FOR IMPLANTING INTRAOCULAR PRESSURE SENSOR

(75) Inventors: Vicente Diaz, New Haven, CT (US); Amir Cohen, Livingston, NJ (US)

(73) Assignee: YALE UNIVERSITY, New Haven, CT (US)

(21) Appl. No.: 12/935,274

(22) PCT Filed: Apr. 17, 2009
(86) PCT No.: PCT/US09/40935

§ 371 Date: Dec. 10, 2010

(30) Foreign Application Priority Data
Apr. 17, 2008 (US) 61124628
Oct. 6, 2008 (US) 61195304

(51) Int. Cl. A61B 3/00

(52) U.S. Cl. 600/558

(57) ABSTRACT

Methods of implanting an intraocular pressure sensor and systems tbr sensing intraocular pressure are disclosed. An intraocular pressure sensor, including, a top surface, may be placed through the sclera of an eye of a patient. The intraocular pressure sensor may be caused to be inserted into the sclera until the top surface of the intraocular pressure sensor is substantially flush with the exterior wall of the sclera. An implantation wand may be used to assist in the insertion process. The wand may be disengaged from the tntraocuiar pressure sensor after it has been implanted.

### CROSS-REFERENCE TO RELATED APPLICATIONS

This application claims priority to pending U.S. Provisional Application Ser. No. 61/124,628 to Diaz et al., entitled “Method for Treating Glaucoma with Digital Data Monitoring,” filed Apr. 17, 2008 and U.S. Provisional Application Ser. No. 61/195,304 to Diaz et al., entitled “Method for Implanting Intraocular Pressure Sensor,” filed Oct. 6, 2008. Each of these applications is incorporated herein by reference in its entirety.

Not Applicable

### BACKGROUND

Glaucoma is a group of diseases that affect the optic nerve causing a loss of retinal ganglion cells in a characteristic pattern of optic neuropathy. The primary function of the optic nerve is to convey visual signals received by the retina to the brain. Individuals suffering from glaucoma typically experience a build-up of aqueous fluid, which can cause the pressure inside the eye (i.e., the intraocular pressure) to rise. Elevated intraocular pressure (IOP) is one of the most significant risk factors for developing glaucoma and is the main metric that is presently addressed when trying to treat the disease. As retinal ganglion cells are damaged by glaucoma, the visual signals from at least a portion of visual field are no longer reported to the brain and a blind spot (i.e., a scotoma) is formed. As glaucoma progresses and more nerve tissue is lost, the scotoma can increase in size and/or additional scotomas can form. Untreated glaucoma is the second leading cause of blindness worldwide, affecting one in two hundred people under the age of fifty and 10% of those over the age of eighty.

Intraocular pressure is largely affected by the amount of aqueous fluid entering and exiting the eye. Typically, IOP in a human eye is between approximately 10 mmHg and 20 mmHg. Individuals that suffer from glaucoma often have an IOP that is dramatically higher, or which varies more than normal.

Aqueous fluid is produced by a ciliary body and is intended to flow between the iris and the lens, through the pupil and to the drainage angle at the junction of the iris and the cornea. The aqueous fluid then exits the eye through a tissue called the trabecular meshwork in the drainage angle. As the aqueous fluid passes through the eye, it is intended to supply the lens and cornea with nutrients and can away waste products. If the aqueous fluid is produced faster than it drains, the intraocular pressure will rise.

Two major types of glaucoma are associated with an elevated IOP: open-angle glaucoma and closed-angle glaucoma. In open-angle glaucoma, the drainage angle between the cornea and the iris is open and allows the aqueous fluid of the eye to reach the trabecular meshwork, the principal location for fluid drainage. Individuals having open-angle glaucoma typically have abnormalities in the trabecular meshwork that reduce the outflow of aqueous fluid from the eye. In closed-angle glaucoma, the trabecular meshwork is visibly obstructed and the aqueous fluid is substantially prevented from draining out of the eye.

Although IOP is only one major risk factor for glaucoma, the progression of glaucoma can be substantially halted in some patients when IOP is lowered to a normal level using, for example, medicines or surgical procedures. In other patients, the optic nerve can be injured at non-elevated pressures (i.e., pressures in the normal range). The optic nerve of such a patient (with normal tension glaucoma) generally appears to be sensitive to damage at intraocular pressures that most people tolerate without any harmful effects. Studies have shown that lowering IOP to low-normal or even sub-normal pressures can halt the progression of normal tension glaucoma.

Medicines that currently serve as mainstays for glaucoma treatment generally reduce IOP either by suppressing aqueous fluid production or by facilitating drainage of aqueous fluid from the eye. Alternately, laser surgical procedures are used to treat glaucoma by attempting to stimulate the trabecular meshwork to work more effectively.

A trabeculectomy is a surgical procedure that is used to lower IOP. When performing a trabeculectomy, a scleral flap is created to allow aqueous fluid to drain from the anterior chamber without deflating the eye. Fluid can then flow out onto the surface of the eye and underneath the conjunctiva (i.e., the transparent membrane that lines the sclera and the eyelids).

Another surgical procedure places valve implants (also known as aqueous shunts or glaucoma drainage devices), such as the Molteno, Baerveldt, and Ahmed tubes, into the eye to drain aqueous fluid into the sub-conjunctival space. Valve implants have proven to be of assistance to reducing IOP particularly in patients that are at high risk for failing with a standard trabeculectomy. An exemplary valve implant placed in the suprachoroidal space that includes an intraocular pressure sensor is disclosed in U.S. Patent Application Publication No. 2009/0069648 to Irazoqui et al.

However, problems can result from the use of valve implants. For example, implantation procedures for current valve implants require a major surgical procedure to be performed (i.e., a surgical procedure requiring general anesthesia and partial dissection of the eye to access the suprachoroidal space). As such the risk of vision impairment, reaction to anesthesia or the like from such procedures is non-negligible.

Tonometry is a procedure used by eye-care professionals to determine intraocular pressure. Although, as stated above, glaucoma is not diagnosed solely on the basis of elevated IOP, its accurate measurement is often useful for diagnosis and follow-up treatment for glaucoma.

A number of different types of error can significantly influence the accuracy of a tonometry procedure. Potential errors may be related, without limitation, to the following factors: concentration of fluoresce in the precorneal tear film, the degree of fluorescence, the width of tear meniscus, alignment of semicircles, corneal thickness, conical curvature, orientation of tonometer prisms (in astigmatic patients), duration of contact between the cornea and the tonometer prisms, and repeated tonometry within a limited period of time. In addition, corneal thickness and rigidity can have an effect on the measured value of the IOP. Moreover, some forms of refractive surgery (e.g., photorefractive keratectomy) may cause traditional IOP measurements to appear normal when the IOP is in fact abnormally high.

The fluctuation of IOP over the course of a day is termed the diurnal variation. Typically, LOP demonstrates a 3 to 6 mmHg diurnal variation for normal eyes. The diurnal variation in IOP for patients with glaucoma is often substantially higher. In fact, the amount of diurnal variation can itself be a risk factor for the progression of glaucoma.

Several physiological influences may affect IOP, such as a patient's heart rate, respiration, exercise, fluid intake, systemic medication and topical drugs. Alcohol consumption can lead to a transient decrease in IOP, and caffeine intake can result in an increase in IOP. It is presently very difficult to identify diurnal variation in IOP using conventional techniques. When alerted to a loss in vision that cannot be explained by a static IOP measurement, an eye-care professional may schedule a patient for a plurality of visits at various times of the day in order to approximate diurnal variation. However, this is less than ideal for a variety of reasons. For example, it may not be feasible for a patient and/or a physician to evaluate IOP at various times of the day. Moreover, transient variations in IOP within a given day can still go undetected.

A patient's behavior can also reduce the effectiveness of treating glaucoma. For example, ophthalmologists currently base treatment decisions on IOP measurements obtained during office visits. It is assumed that the patient's average IOP reflects the reading obtained in the doctor's office. However, many factors can cause this assumption to be incorrect. For example, patient non-compliance can cause improper readings (e.g., the patient may only take prescribed medicines on days immediately preceding their appointment). Also, studies have shown that activities such as practicing yoga, scuba diving and playing wind instruments can cause large fluctuations in IOP. Not knowing about such activities can confound a doctor in trying to explain progressive vision loss.

### SUMMARY

Before the present systems, devices and methods are described, it is to be understood that this disclosure is not limited to the particular systems, devices and methods described, as these may vary. It is also to be understood that the terminology used in the description is for the purpose of describing the particular versions or embodiments only, and is not intended to limit the scope.

It must also be noted that as used herein and in the appended claims, the singular forms “a,” “an,” and “the” include plural references unless the context clearly dictates otherwise. Thus, for example, reference to a “sensor” is a reference to one or more sensors and equivalents thereof known to those skilled in the art, and so forth. Unless defined otherwise, all technical and scientific terms used herein have the same meanings as commonly understood by one of ordinary skill in the art. Although any methods, materials, and devices similar or equivalent to those described herein can be used in the practice or testing of embodiments, the preferred methods, materials, and devices are now described. All publications mentioned herein are incorporated by reference. Nothing herein is to be construed as an admission that the embodiments described herein are not entitled to antedate such disclosure by virtue of prior invention. As used herein, the term “comprising” means “including, but not limited to.”

In an embodiment, a method of implanting an intraocular pressure sensor may include placing an intraocular pressure sensor, including a top surface, through the sclera of an eye of a patient, and causing the intraocular pressure sensor to be inserted into the sclera until the top surface of the intraocular pressure sensor is substantially flush with the exterior wall of the sclera. The intraocular pressure sensor further includes a coil, screw, or flare disposed underneath the top surface.

In an embodiment, a method of implanting an intraocular pressure sensor may include determining an insertion location on a sclera of an eye of a patient, loading an intraocular pressure sensor, including a top surface and a coil disposed underneath the top surface, in an implantation wand, placing the coil of the intraocular pressure sensor at the insertion location, rotating the implantation wand to cause the intraocular pressure sensor to be inserted at the insertion location mad the top surface of the intraocular pressure sensor is substantially flush with the exterior wall of the sclera, and disengaging the implantation wand from the intraocular pressure sensor. An external surface of the intraocular pressure sensor may include a bio-inert surface coating or at least a portion may include a porous cellular ingrowth coating.

### BRIEF DESCRIPTION OF THE DRAWINGS

Aspects, features, benefits and advantages of the present invention will be apparent with regard to the following description and accompanying drawings, of which:

FIG. 1 depicts an exemplary intraocular pressure sensor and an exemplary implantation wand according to an embodiment.

FIG. 2 depicts a block diagram of an exemplary pressure-sensing device for use with an intraocular pressure sensor according to an embodiment.

FIG. 3 depicts a flow diagram of an exemplary method for inserting an intraocular pressure sensor according to an embodiment.

FIG. 4 depicts a cross-sectional view of an exemplary intraocular pressure sensor inserted into the sclera of a human eye.

### DETAILED DESCRIPTION

The present disclosure discloses embodiments that are not subject to the potential sources of error in determining IOP discussed above because devices incorporating the features described herein can calculate LOP using a more-accurate and less-invasive procedure. For example, LOP readings using the disclosed device are not compromised by factors such as cortical thickness and/or topography, dye or tear film.

Moreover, the present disclosure describes devices and features that can eliminate problems with respect to monitoring diurnal variation by recording multiple LOP data points throughout the day. As such, physicians can be provided with a more accurate measure of diurnal variation in IOP.

Furthermore, the present disclosure teaches devices and features that can alert a healthcare professional when a patient's IOP is abnormal. This may enable the healthcare professional to work with a patient to avoid activities and/or behaviors that put them at risk for vision loss.

The devices and features disclosed herein provide ophthalmologists with methods and systems fir more effectively treating glaucoma and reducing the occurrence of permanent vision toss. Devices disclosed herein measure IOP in a manner that is free from all sources of error involved with corneal applanation. Because IOP readings are being stored periodically and/or continuously, physicians are enabled to non-invasively and unobtrusively measure the variation and range of IOP for a patient. In turn, this can enable more appropriate delivery of pharmaceutical and/or surgical care for glaucoma patients.

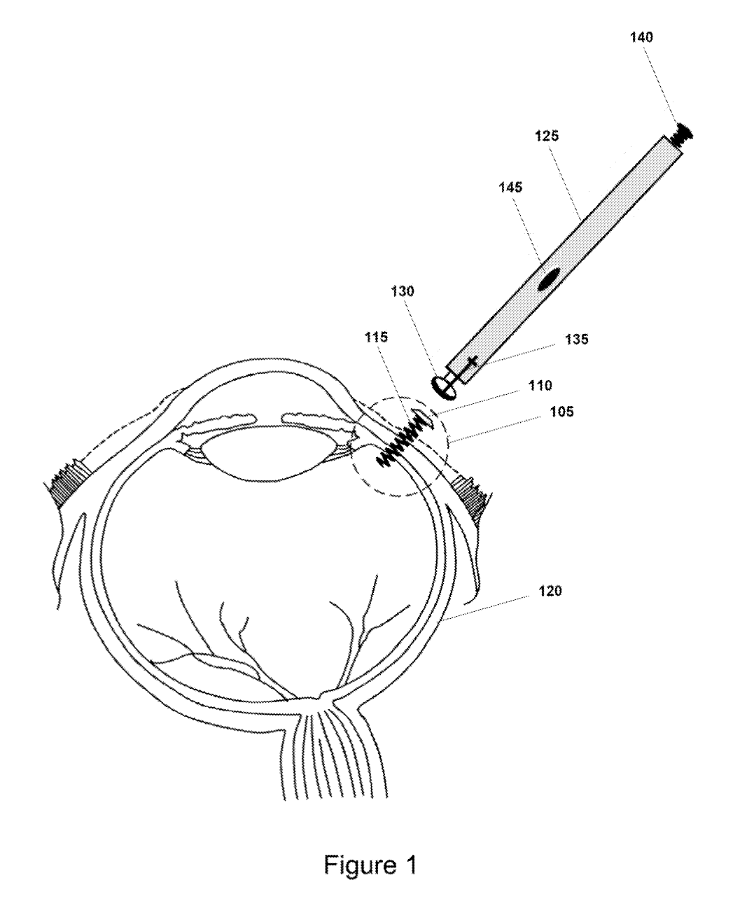

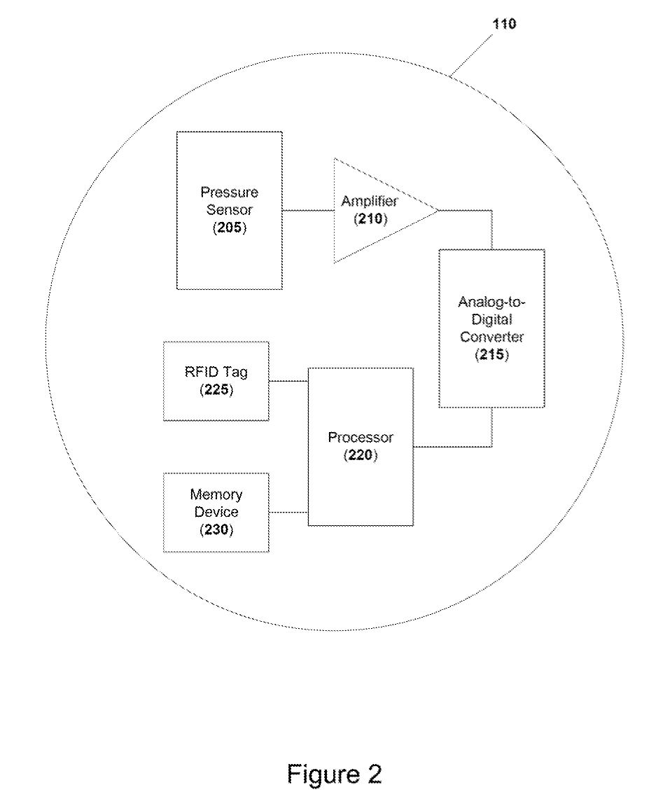

FIG. 1 depicts an exemplary intraocular pressure sensor and an exemplary implantation wand according to an embodiment. As shown in FIG. 1, the intraocular pressure sensor 105 may include a pressure-sensing device 110 and a coil 115. In an embodiment, the intraocular pressure sensor 105 may have a top surface having, a head diameter of approximately 2 millimeters, a coil diameter of approximately 1 millimeter and a depth of approximately 3 millimeters. Exemplary components of a pressure-sensing device 110 are described in more detail with respect to FIG. 2 below. The coil 115 may have a corkscrew shape that assists with insertion of the intraocular pressure sensor 105 into an eye, such as 120. In an embodiment, the coil 115 may have a tapered tip which is configured to engage with the tissue of an eye when the intraocular pressure sensor 105 is implanted. The tapered tip of the coil 115 may substantially prevent the intraocular pressure sensor 105 from being unintentionally withdrawn from the eye. In embodiments, the intraocular pressure sensor may include a screw, a flare, or the like, rather than a coil.

As further shown in FIG. 1 the implantation wand 125 may have a ring-Shaped base 130 that is intended to make contact with the eye. The base 130 may be used to hold the intraocular pressure sensor 105 during an insertion process. The base 130 is attached to, for example, a spring mechanism 135, which provides resistance as the intraocular pressure, sensor 105 approaches the surface of the eye. The distal end of the wand 125 may include a knob 140 that is used to twist the intraocular pressure sensor 105 causing it to move into the eye by a specified distance per turn. In an embodiment, the distance per turn may be related to the distance between adjacent portions of the coil 115. In an embodiment, the distance per turn may be approximately 500 microns. In an embodiment, the wand 125 may include a button 145 that disengages the intraocular pressure sensor 105 from the wand.

FIG. 2 depicts a block diagram of an exemplary pressure-sensing device for use with an intraocular pressure sensor according to an embodiment. As shown in FIG. 2, the pressure-sensing device 110 may include a pressure sensor 205, an amplifier 210, an analog-to-digital converter 215, a processor 220 and a Radio Frequency Identification (RFID) tag 225. The pressure sensor 205 is configured to measure pressure when, for example, the intraocular pressure sensor 105, including the pressure sensor, is inserted within an eye. An exemplary pressure sensor 205 is the MS7212A pressure sensor from Measurement Specialties of Fremont, Calif. In an embodiment, the pressure sensor 205 may output differential voltage signals based on the measured pressure. An amplifier 210 may optionally amplify the differential voltage, signals output by the pressure sensor 205. An analog-to-digital converter 215 may convert the (amplified) differential voltage signals from analog signals to digital signals. The digital signals may identify a digital value that is representative of the difference between the differential voltage signals. The digital signals may be provided to the processor 220. The processor 220 may process the received information and provide data to the RED tag 225 for transmission to a remote device. In an embodiment, the pressure-sensing device 110 may further include, a memory device 230 for storing values of the digital signals and associated time stamp information. The memory device 230 may receive the digital signal values from the processor 220 and may be used to store such values prior to transmission using the RFID tag 225.

In an embodiment, the components of the pressure-sensing device 110 may be placed on a substrate and have at least a portion coated with a coating. The substrate may be made of silicon or any other insulating substance that does not react with the human body. Similarly, the coating may be designed to be non-reactive when placed in the human body. An appropriate coating is configured to protect the components of the pressure-sensing device 110 from the environment in which the pressure-sensing device is placed. An exemplary coating useful for such a purpose may include monochloro-p-xylelene (Parylene C). Additional coatings may include a bio-inert coating or a porous cellular ingrowth coating. Alternate or additional substrates ardor coatings may be used within the scope of this disclosure. In other embodiments, the intraocular pressure sensor may have a least a portion of an outer surface having, a bio-inert coating or a porous cellular ingrowth coating.

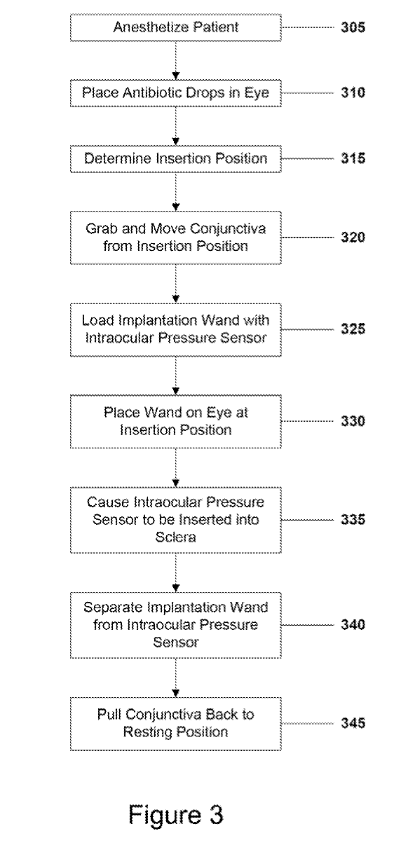

FIG. 3 depicts a flow diagram of an exemplary method for implanting an intraocular pressure sensor according to an embodiment. As shown in FIG. 3, a patient is anesthetized 305 and antibiotic drops are placed 310 in the eye as a prophylactic measure. The patient is told to look down once anesthetization is complete in order to expose the proper portion of the eye. Various methods of anesthetization can be used within the scope of this disclosure, although local anesthetic is preferred to avoid medical complications inherent with the use of systemic anesthesia.

An insertion position in the eye is determined 315 with respect to the corneal limbus. In an embodiment, the determined insertion position is approximately 3.5 millimeters from the limbus in a pseudophakic eye. In an alternate embodiment, the determined insertion position is approximately 4 millimeters from the limbus in a phakic eye. Because the diameter of the wand's footplate may be sized to conform to the size of the intraocular pressure sensor (i.e., the diameter of the base of the wand may be approximately 2 millimeters), the edge of the wand base may be placed 2.5 millimeters from the limbus for a pseudophakic eye and 3 millimeters from the limbus for a phakic eye in order to achieve the desired insertion positing. In an embodiment, the insertion position is determined 315 using calipers. Other measuring devices may also be used within the scope of this disclosure.

In an embodiment, forceps may be used to grab 320 the conjunctiva at the insertion position. After the conjunctiva has been grabbed 320, the conjunctiva can be pulled towards the corneal limbus. Other devices may also be used to grab 320 and pull the conjunctiva if desired.

The implantation wand is loaded 325 with an intraocular pressure sensor and placed 330 on the eye with the anterior edge of the ring-shaped base abutting the insertion position. The contact between the base of the implantation wand and the surface of the eye at the insertion position serves to stabilize the wand and ensure perpendicular implantation of the intraocular pressure sensor. In an embodiment, a physician may apply pressure to the top portion of the implantation wand. The applied pressure causes the intraocular pressure sensor to be forced downward within the implantation wand. Preferably, additional pressure may be applied until the coil of the intraocular pressure sensor abuts the surface of the eye.

The physician then turns 335 a knob on the top portion of the implantation wand. Turning 335 the knob may cause the intraocular pressure sensor to rotate in a manner such that it is inserted into the sclera of the eye at the insertion position. The physician may turn 335 the knob until the top of the intraocular pressure sensor is flush with the exterior wall of the sclera. The number of turns may be determined based on the length of the coil of the intraocular pressure sensor and the depth which the sensor moves into the eye with each turn. Alternately, the physician may examine the depth to which the intraocular pressure sensor has been inserted while performing the implantation procedure.

The implantation wand is then separated 340 from the intraocular pressure sensor. In an embodiment, a button or other mechanism on the implantation wand may be activated to separate 340 the wand from the intraocular pressure sensor.

The forceps are then used to pull 345 the conjunctiva off of the intraocular pressure sensor and back to a resting position.

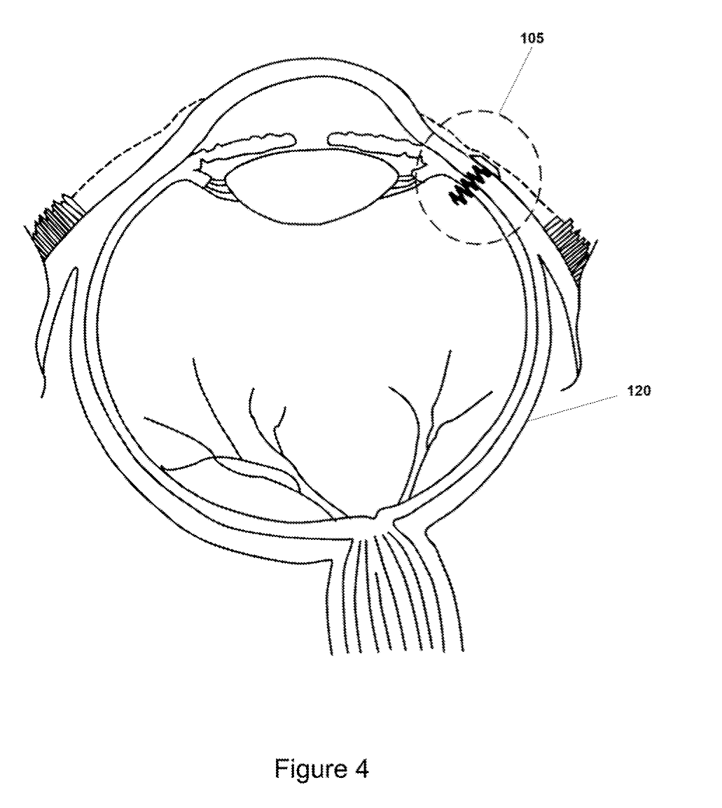

Upon completion of the implantation procedure, the intraocular pressure sensor may be inside the eye and firmly anchored within the sclera. The intraocular pressure sensor may be completely covered by the conjunctiva with the conjunctival entry point lying safely underneath the lid. FIG. 4 depicts a cross-sectional view of an exemplary intraocular pressure sensor inserted into the sclera of a human eye.

Implanting an intraocular pressure sensor according to the method described above (or similar methods) has numerous advantages over conventional procedures. For example, because a physician can readily access the scleral region of a patient's eye, the use of the procedure disclosed herein (or similar procedures) obviates the need for the use of general anesthetic, systemic sedation, or in-patient surgical procedures requiring dissection of the eye. As a result, morbidity and/or medical costs to the patient and/or the healthcare system are reduced. Moreover, the occurrence of ocular inflammation, which can occur during valve implantation, is substantially reduced.

In an embodiment, once the device is implanted in the patient's eye, the device may wirelessly transmit, information pertaining to the IOP on a continuous or periodic basis to a remote device. The wireless transmission may be performed using standard radio frequency identification (RFID) protocols. In an embodiment, the remote device may be located within a bracelet or other adornment worn by the patient. The remote device may store the information received from the intraocular pressure sensor until the patient next visits the physician administering treatment to the patient. Alternately, the remote device may transmit the information via a computer network, such as the Internet, to a physician's office for teal-time assessment of the patient's intraocular pressure.

In an alternate embodiment, the intraocular pressure sensor may include a memory device that stores the intraocular pressure information locally. The intraocular pressure information may be transmitted periodically to a remote device or to a physician's office via a computer network.

It will be appreciated that one or more of the above-disclosed and other features and functions, or alternatives thereof, may be desirably combined into many other different systems or applications. It will also be appreciated that various presently unforeseen or unanticipated alternatives, modifications, variations or improvements therein may be subsequently made by those skilled in the art which are also intended to be encompassed by the disclosed embodiments.

What is claimed is:

1. A method of implanting an intraocular pressure sensor, the method comprising:
placing an intraocular pressure sensor through the sclera of an eye of a patient, wherein the intraocular pressure sensor comprises a top surface; and
causing the intraocular pressure sensor to be inserted into the sclera until the top surface of the intraocular pressure sensor is substantially flush with the exterior wall of the sclera.

2. The method of claim 1 wherein the intraocular pressure sensor further comprises a coil disposed underneath the top surface, and wherein causing, the intraocular pressure sensor to be inserted comprises rotating the intraocular pressure sensor in a direction that causes the coil to pass through the sclera.

3. The method of claim 2 wherein the coil comprises a tapered tip configured to engage with tissue of the eye and substantially prevent the intraocular pressure sensor from being withdrawn from the eye after the intraocular pressure sensor has been inserted in the eye.

4. The method of claim 1 further comprising:
determining an insertion location on the sclera at which the intraocular pressure sensor is placed for insertion.

5. The method of claim 4 wherein the insertion location for a pseudophakic eye is approximately 3.5 millimeters from the limbus.

6. The method of claim 4 wherein the insertion location for a phakic eye is approximately 4 millimeters from the limbus.

7. The method of claim 1 wherein the intraocular pressure sensor further comprises a screw disposed underneath the top surface, and wherein causing the intraocular pressure sensor to be inserted comprises rotating the intraocular pressure sensor in a direction that causes the screw to pass through the sclera.

8. The method of claim 1 wherein the intraocular pressure sensor further comprises a flare disposed underneath the top surface, and Wherein causing the intraocular pressure sensor to be inserted comprises moving the intraocular pressure sensor in a direction that causes the flare to pass through the sclera.

9. A method of implanting an intraocular pressure sensor, the method comprising;
determining an insertion location on a sclera of an eye of a patient;
loading an intraocular pressure sensor in an implantation wand, wherein the intraocular pressure sensor comprises a top surface and a coil disposed underneath the top surface;
placing the coil of the intraocular pressure sensor at the insertion location;
rotating the implantation wand to cause the intraocular pressure sensor to be inserted at the insertion location until the top surface of the intraocular pressure sensor is substantially flush with the exterior wall of the sclera; and
disengaging the implantation wand from the intraocular pressure sensor.

10. The method of claim 9 wherein the coil comprises a tapered tip configured to engage with tissue of the eye and substantially prevent the intraocular pressure sensor from being withdrawn from the eye after the intraocular pressure sensor has been inserted in the eye.

11. The method of claim 9 wherein the insertion location for as pseudophakic eye is approximately 3.5 millimeters from the limbus.

12. The method of claim 9 wherein the insertion location for a phakic eye is approximately 4 millimeters from the limbus.

13. The method of claim 9 wherein the intraocular pressure sensor comprises an outer bio-inert surface coating.

14. The method of claim 9 wherein at least a portion of an external surface of the intraocular pressure sensor comprises a porous cellular ingrowth coating.
